(19) United States
(12) Patent Application Publication
(10) Pub. No.: US 2004/0187196 A1
(43) Pub. Date: Sep. 30, 2004

(54) Nose-bridge for swimming goggles

(76) Inventor: Joseph Haslbeck, West Vancouver (CA)

Correspondence Address: CHRISTINE, PARKER & HALE, LLP, P.O. BOX 7068, PASADENA, CA, 91109-7068, US

(21) Appl. No.: 10/400,757

(22) Filed: Mar. 26, 2003

(51) Int. Cl. A61F 9/02

(52) U.S. Cl. 2/426

(57) ABSTRACT

The present invention related to a nose-bridge for swimming goggles and swimming goggles having the nose-bridge of the present invention. The swimming goggles of the present invention include two eyepieces, each eyepiece accommodating a lens and having an inner end, each of the inner ends of the swimming goggles having a portion defining a hole therethrough. The nose bridge includes a strap for joining the inner ends of the eyepieces. The strap has first and second end portions and is slidably passable through each of the holes. In some embodiments, the nose bridge further includes a connector for connecting the first and second end portions when the strap is passed through the holes so that the strap forms a closed loop. The connector thereby allows for adjustment of the strap to vary the circumference of the loop and thereby adjust the distance between the eyepieces. In other embodiments, the swimming goggles further include a set of protrusions near each hole on the inner end, each of the set of protrusions extending towards each other to define an opening between the set of protrusion, such that when an end of the strap is passed through one of the holes and moved between one of the set of protrusions into one of the openings, the strap is resiliently held in place in the opening, allowing for adjustment of the distance between the eyepieces.

### FIELD OF THE INVENTION

This invention relates to swimming goggles, and, more particularly, to a nose-bridge for swimming goggles.

### BACKGROUND OF THE INVENTION

Swimming goggles have been known for many years. One conventional type of swimming goggles includes two eyepieces which are interconnected by a relatively thin and flexible plastic strap, which serves as a nose-bridge, and a headband which passes around the wearer's head to hold the swimming goggles on the face.

Attempts have been made to allow for adjustment of the nose-bridge to account for varying shapes and sizes of a swimmer's face, so that the eyepieces are always comfortably and securely located over the wearer's eyes. This is often accomplished by allowing for adjustment of the length of the nose-bridge thereby altering the distance between the two eyepieces. In an attempt to improve the adjustability of the nose-bridge between the eyepieces, various different types of swimming goggles have been proposed.

For example, swimming goggles have been disclosed wherein the nose-bridge strap has opposite outer ends which are received in complementary openings provided in peripheral rims surrounding the lenses of the eyepieces. The outer ends of the strap have “barb-like” steps which engage complementary projections or edges of the openings in the peripheral rims. To adjust the nose-bridge for persons having different eye spacing, at least one of the outer ends of the strap are moved in or out of the respective opening to permit different barb-like steps to engage the projection or edge of the opening, allowing for incremental adjustment of the distance between the eyepieces. U.S. Pat. No. 5,459,882 (Yamamoto) discloses an example such a nose-bridge strap with barb-like steps at outer ends thereof.

One of the problems with such nose-bridge straps is that, in order to permit easy adjustment of the strap within the opening, there must be provided adequate clearance between the strap and the projection or edge of the opening to facilitate engagement and disengagement of the barb-like steps. This allows for excessive movement between the strap and the opening which can cause instability of the eyepieces engaging the wearer's face. While it is possible to reduce this instability to some extent by tightening the headband, excessive tightening of the headband causes increased discomfort for the wearer of the goggles. The clearance between the strap and the opening can also cause problems when the goggles are removed from the wearer's head. When the strap is not subject to the headband tension, random movements of the goggles can cause inadvertent movement between the strap and the opening which can disengage the barb or step from the edge of the opening, thus readjusting the eyepiece spacing. Another problem with such a nosepiece is the inherent limitation in varying the distance between the eyepieces, given that the barb-like steps are set specific distances apart. Such a system does not allow for infinite adjustability between the eyepieces.

To avoid use of the system which incorporates a nose-bridge having “barb-like” steps, various other types of systems have been devised to locate eyepieces securely against the face, while permitting for adjustment of the spacing therebetween. For example, various swimming goggle models have been proposed wherein the eyepieces are connected together by lengths of string passing through openings adjacent inner portions of the eyepieces. See for example U.S. Pat. No. 5,502,844 (Alvarado). Other swimming goggles use string which extends across from eyepiece to eyepiece and wraps around the tops and bottoms of the eyepieces. This allows for adjustment of the length of the nose-bridge at either the outer edges of the eyepieces or by adjustment of the headband itself. See for example, U.S. Pat. No. 6,119,277 (Chiang), U.S. Pat. No. 6,247,187 (Chiang), U.S. Pat. No. 6,349,417 (Chiang) or U.S. Pat. No. 6,505,352 (Chiang). One of the problems of such swimming goggles is that the nose-bridges would tend to interfere with the wearer's nose, since the string would be stretched straight from eyepiece to eyepiece. Additionally, the string disclosed in such swimming goggles would tend to apply a static force towards the nose, which does not ensure a continuous seal when the wearer moves his or her facial muscles.

Another type of nose-bridge structure that has been proposed is one where the nose-bridge is an elongated member connected between two eyepieces of the swimming goggles and each of the eyepieces has a through hole provided on an inner side thereof, which face each other. The elongated member nose-bridge is made up of a flexible tubing having two end sections respectively extending through the holes of the eyepieces with a central section connecting the eyepieces and defining a distance therebetween. Each of the end sections of the tubes contains a bead-like fastener moveable within the tube, which releasably holds the tubing relative to the hole of each eyepiece. The bead-like fastener is moveable along the tube by squeezing the tube, thereby allowing for the distance defined by the central section of the tubing to be adjusted. Such a nose-bridge is disclosed in U.S. Pat. No. 5,894,605. The problem with such a nose-bridge is that the bead-like fastener is moveable along the tubing, and it is possible for the distance between the eyepieces to be undesirably adjusted if the bead is displaced along the tubing.

In another type of swimming goggle, there had been disclosed a nose-bridge rigidly connected to the eyepieces, wherein the nose-bridge may be twisted to shorten the distance between the eyepieces. For example, U.S. Pat. No. 6,199,279 (Haslbeck) discloses such a model. One of the difficulties with such a nose-bridge is that each twist of the nose-bridge will vary the distance between the eyepieces in a fixed manner, and will not allow for minute adjustments. Another problem with such a swimming goggle is that the nose-bridge must be rigid enough to create an arch between the eyepieces to prevent the nose-bridge from contacting the wearer's nose. This rigidity results in a static force towards the nose, which does not ensure a continuous seal when the wearer moves his or her facial muscles. Further, the rigidity of the nose-piece combined with the inherent limitations in adjustability of the nose-bridge length can create difficulties with providing a perfectly sealed fit, since a rigid nose-bridge which is too long and which arches away from the wearer's face, will tend to pull the inner edges of the eyepieces away from the wearer's face. Similarly, a rigid nose-bridge which is too short and arches away from the wearer's face, will tend to pull the outer edges of the eyepieces away from the wearer's face.

Another problem with the above-described nose-bridges is that they cannot be easily incorporated into existing goggles to improve the fit thereof.

What is desired therefore is a nose-bridge that does not result in excessive movement of the goggle eyepieces, but at the same time allows for automatic adjustment of the eyepieces on the wearer which are necessary to allow for movement of facial muscles. Further, it is desired to have a nose-bridge that is infinitely adjustable and comfortable for the wearer. It is also desirable to have a nose-bridge that can easily be incorporated into existing goggles.

### SUMMARY OF THE INVENTION

In accordance with one aspect of the invention, there is provided a nose-bridge for swimming goggles. The swimming goggles have two eyepieces, each eyepiece accommodating a lens and having an inner end and each of the inner ends having a portion defining a hole therethrough. The nose bridge includes a strap for joining the inner ends of the eyepieces. The strap has first and second end portions and is slidably passable through each of the holes. The nose bridge further includes a connector for connecting the first and second end portions when the strap is passed through the holes so that the strap forms a closed loop. The connector thereby allows for adjustment of the strap to vary the circumference of the loop and thereby adjust the distance between the eyepieces.

In another aspect of the present invention, there is provided swimming goggles. The swimming goggles include two eyepieces, each eyepiece accommodating a lens and having an inner end, and each of the inner ends having a portion defining a hole therethrough. The swimming goggles further include a strap for joining the inner ends to form a nose-bridge therebetween. The strap has first and second end portions and is slidably passable through each of the holes. The swimming goggles further include a connector for connecting the first and second end portions when the strap is passed through the holes, so that the strap forms a closed loop. The connector allows for adjustment of the strap to vary the circumference of the loop and thereby adjust the distance between the eyepieces.

The connector in such embodiments may allow for infinite adjustment of the distance between the eyepieces. Such a connector may include a portion defining at least one aperture, through which the strap is slidably passable, and two sets of protrusions. Each set of protrusions extends towards each other to define an opening between the set of protrusions, such that when an end portion of the strap is passed through the aperture and moved between one of the set of protrusions into one of the openings, that end portion is resiliently held in place in that opening.

In accordance with another aspect of the invention, there is provided a nose-bridge for swimming goggles. The swimming goggles have two eyepieces, each eyepiece accommodating a lens and having an inner end. The nose-bridge comprises a strap for joining the inner ends of the eyepieces, and two connectors attachable to the inner ends of the eyepieces, each connector defining a hole, through which the strap is slidably passable, and a set of protrusions near each hole. Each of the protrusions extend towards each other to define an opening between the set of protrusions, such that when an end portion of the strap is passed through one of the holes and moved between one of the set of protrusions into one of the openings, the end portion is resiliently held in place in the opening.

In yet another emobodiment of the present invention, there is provided swimming goggles. The swimming goggles include two eyepieces, each eyepiece accommodating a lens and having an inner end. Each of the inner ends has a portion defining a hole therethrough. The swimming goggles also include a strap for joining the inner ends to form a nose-bridge therebetween. The strap is slidably passable through each of the holes. The swimming goggles further include a set of protrusions near each hole on the inner end, each of the set of protrusions extending towards each other to define an opening between the set of protrusion, such that when an end of the strap is passed through one of the holes and moved between one of the set of protrusions into one of the openings, the strap is resiliently held in place in the opening.

In some embodiments of the present invention, the strap is elastic.

### BRIEF DESCRIPTION OF THE DRAWINGS

FIG. 1 is a perspective view of swimming goggles having a nose-bridge according to one embodiment of the present invention;

FIG. 2 is a top view of the swimming goggles of FIG. 1;

FIG. 3 is a perspective view of the swimming goggles of FIG. 1, wherein the nose-bridge is partially undone;

FIG. 4 is a perspective view of the swimming goggles of FIG. 1, wherein the nose-bridge is partially undone;

FIG. 5 is a perspective view of swimming goggles having a nose-bridge according to another embodiment of the present invention;

FIG. 6 is a top view of the swimming goggles of FIG. 5;

FIG. 7 is a perspective view of the swimming goggles of FIG. 5, wherein the nose-bridge is partially undone; and

FIG. 8 is a top view of the swimming goggles of FIG. 5, wherein the nose-bridge is partially undone.

### DETAILED DESCRIPTION

The present invention relates to a nose-bridge for swimming goggles. The present invention also includes swimming goggles having a nose-bridge of the invention.

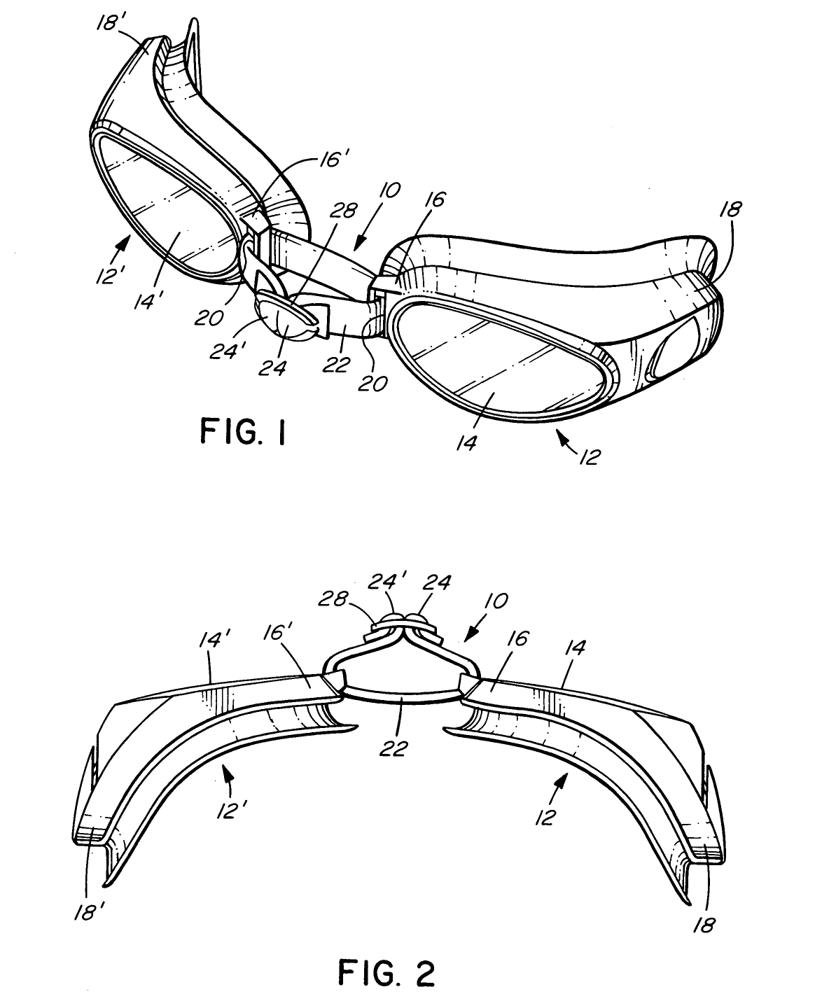

FIGS. 1 through 4 show swimming goggles having a nose-bridge according to one embodiment of the present invention. In the embodiment shown, there is provided a nose-bridge, shown generally at 10, adapted for use on swimming goggles. The swimming goggles for which the nose-bridge is adapted have two eyepieces, 12 and 12′, each accommodating a lens, 14 and 14′. Each eyepiece, 12 and 12′, has an inner end, 16 and 16′, and an outer end, 18 and 18′, and each of the inner ends, 16 and 16′, has a portion defining a hole, 20 and 20′, therethrough. The outer ends, 18 and 18′, are designed to be connected to a headband for holding the swimming goggles in place on the wearer's head.

In the embodiment shown, the nose-bridge 10 includes a strap 22 for joining the inner ends, 16 and 16′. The strap has first and second end portions, 24 and 24′ respectively, and is slidably passable through the holes, 20 and 20′.

The nose-bridge 10 further includes a connector 28, shown in the present embodiment as a buckle-like connector, for connecting the first and second end portions, 24 and 24′, of the strap 22. When the strap 22 is passed through the holes, 20 and 20′, the strap 22 forms a closed loop, and acts as a nosebridge between the two eyepieces, 12 and 12′. The connector 28 allows for adjustment of the strap 22 to vary the circumference of the loop formed by the strap. As the circumference of the loop is shortened or lengthened, the distance between the eyepieces, 12 and 12′, and therefore across the nose-bridge, is shortened or lengthened, respectively.

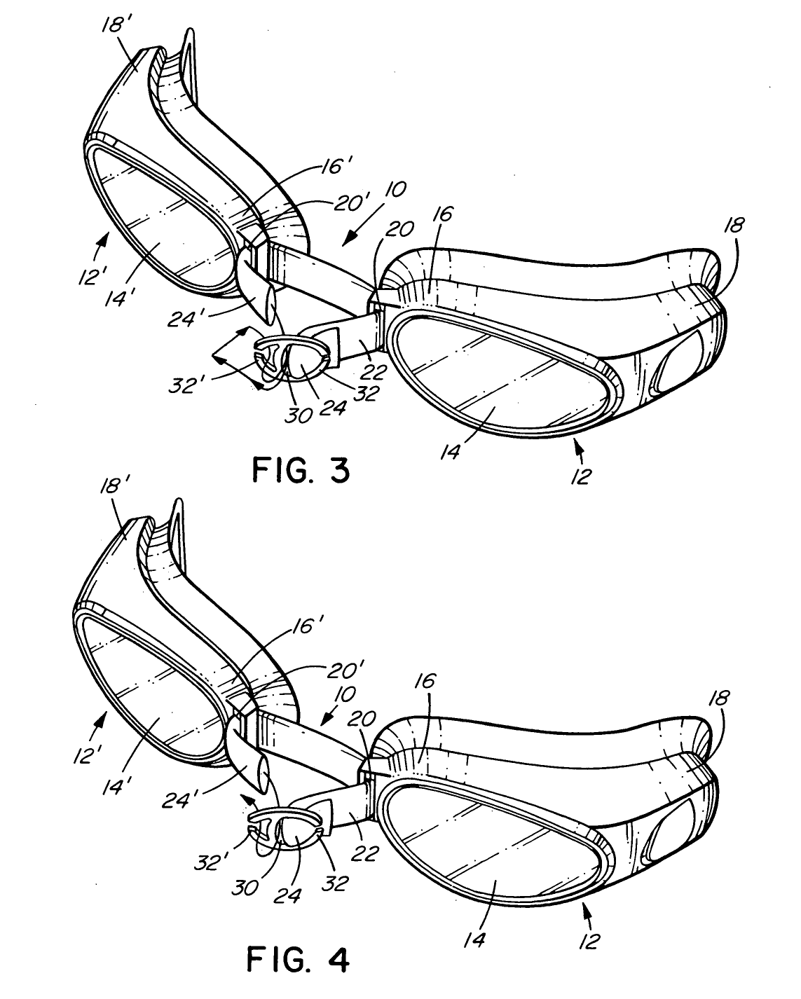

In the embodiment shown in FIGS. 1 through 4, the connector 28 is a buckle-like connector, which allows for infinite adjustment of the strap 22. As can be seen in FIGS. 3 and 4, the connector 28 defines a central aperture 30, and two sets of protrusions, 32 and 32′. Each set of protrusions, 32 and 32′, extend towards each other to define an opening between the set of protrusions, 32 and 32′. In operation, the first and second end portions, 24 and 24′, of the strap 22 are passed through the central aperture 30, which creates the loop which acts as the nose-bridge. The first and second end portions, 24 and 24′, may be pulled through the central aperture 30 to the extent required to create the appropriate distance between the eyepieces, 12 and 12′. The first and second end portions, 24 and 24′, are then pulled or pushed through the two sets of protrusions, 32 and 32′, respectively, and into the openings defined therebetween. FIGS. 3 and 4 illustrate how the end portion 24′ may be moved into the opening defined by the set of protrusions 32′, by either pulling the end portion 24′ through the set of protrusions 32′, or by threading the end portion 24′ into the opening defined by the set of protrusions 32′. The end portions, 24 and 24′, are thereby resiliently held in place in the openings defined by each set of protrusions, 32 and 32′.

It will be recognized that there are various shapes and sizes that the central aperture and sets of protrusions may take in a connector that accomplishes its required function of holding the end portions of the strap in place, while allowing for adjustment of the loop size for varying the distance across the nose-bridge. Further, the central aperture could be comprised of two different apertures for receiving each of the first and second ends of the strap. The shape and size of the aperture and sets of protrusions may be designed to effectively hold the end portions in place, while minimizing the effort required to adjust the circumference of the loop.

For the embodiment of the present invention shown in FIGS. 1 through 4, the nose-bridge can easily be retrofitted onto existing swimming goggles. The nose-bridge can be incorporated into any swimming goggles that have appropriate holes on the inner sides of the eyepieces through which the strap of the nose-bridge is slidably passable.

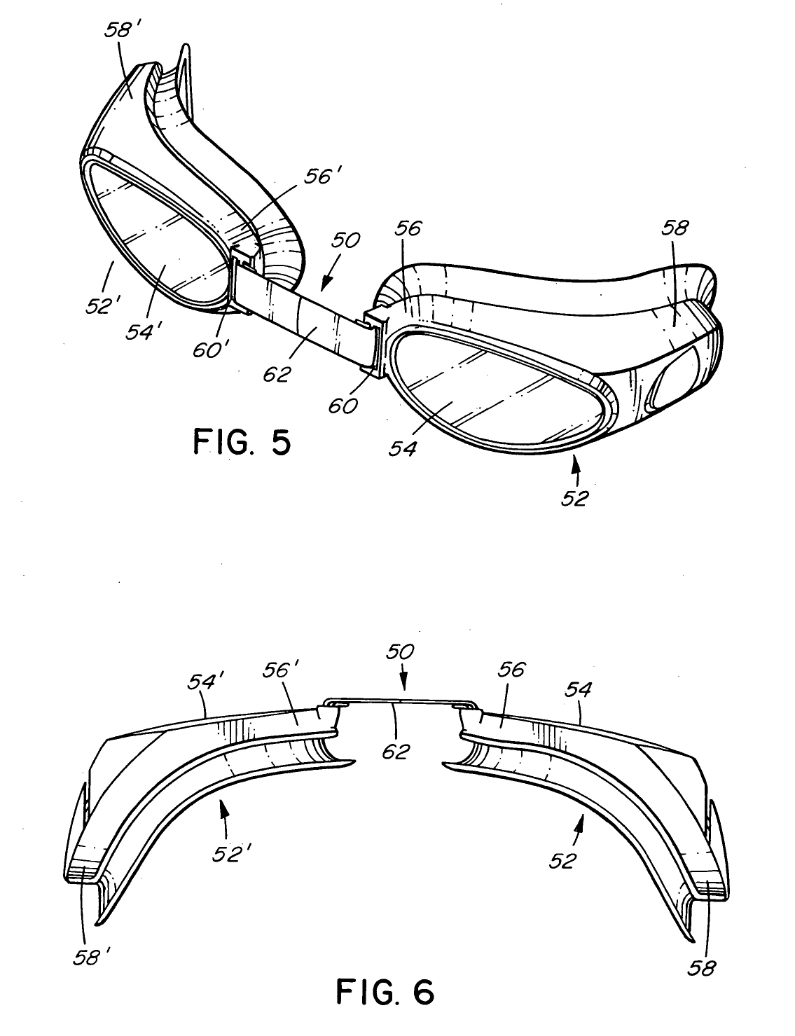

FIGS. 5 through 8 illustrate swimming goggles having a nose-bridge according to a second embodiment of the present invention. In the embodiment shown, there is provided a nose-bridge, shown generally at 50, adapted for use on swimming goggles. As with the previously described embodiment, the swimming goggles for which the nose-bridge is adapted have two eyepieces, 52 and 52′, each accommodating a lens, 54 and 54′. Each eyepiece, 52 and 52′, has an inner end, 56 and 56′, and an outer end, 58 and 58′, and each of the inner ends, 56 and 56′, has a portion defining a hole, 60 and 60′, therethrough. The outer ends, 58 and 58′, are designed to be connected to a headband for holding the swimming goggles in place on the wearer's head.

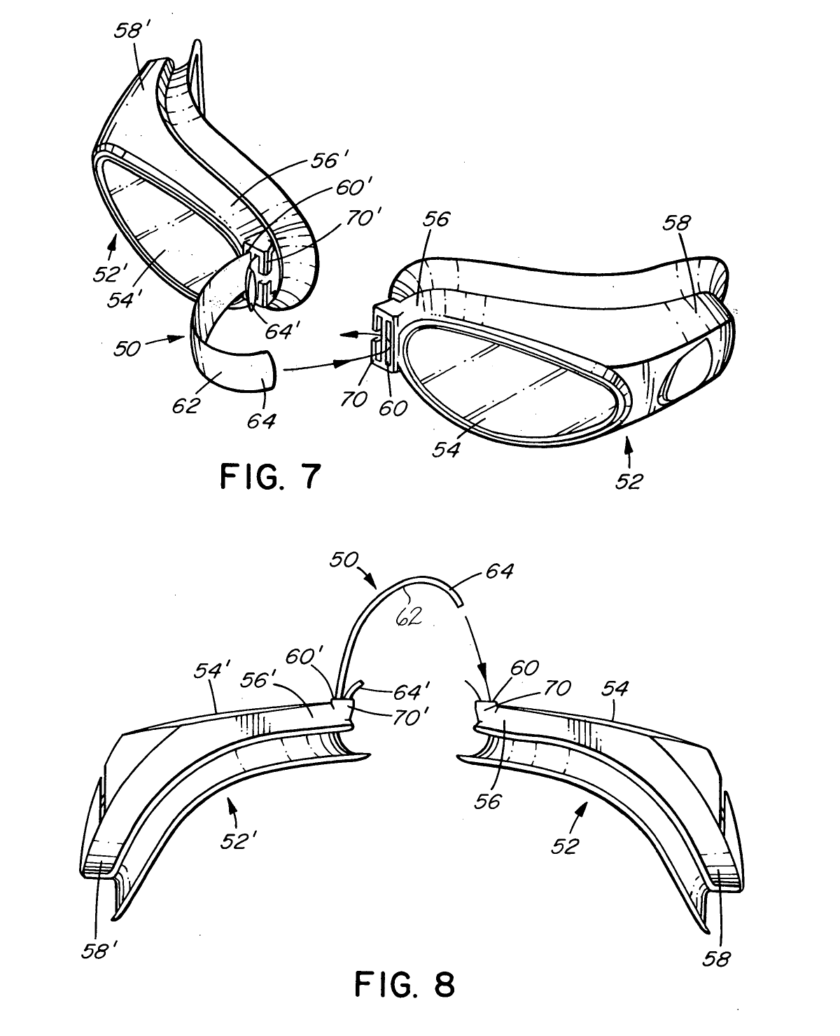

As can be seen in FIGS. 7 and 8, the swimming goggles 8 further include a set of protrusions, 70 and 70′, near each hole, 60 and 60′, on the inner ends, 56 and 56′, of the eyepieces. Each of the set of protrusions, 70 and 70′ extend towards each other to define an opening between each set of protrusions, 70 and 70′.

The nose-bridge 50 includes a strap 62 for joining the inner ends, 56 and 56′, which is slidably passable through the holes, 60 and 60′. The strap has end portions, 64 and 64′. When an end portion, 64 or 64′, is passed through one of the holes, 60 or 60′, and then pushed or pulled between the corresponding set of protrusions, 70 or 70′, into the opening defined by that set of protrusions, the end portion, 64 or 64′, is resiliently held in place in the opening. In operation, each of the end portions, 64 and 64′, of the strap 62 are passed through a respective hole, 60 and 60′, and the end portions, 64 and 64′, are pulled through the holes, 60 and 60′, to the extent required to establish the appropriate distance between the eyepieces, 54 and 54′, by lengthening or shortening the distance of the strap 62 between the eyepieces, 54 and 54′. The end portions, 64 and 64′, are then moved between each respective set of protrusions, 70 and 70′, into the openings defined therebetween. FIGS. 7 and 8 illustrate how the end portion 64 may moved into the opening defined by the set of protrusions 70, by either pulling the end portion 64 through the set of protrusions 70, or by threading the end portion 64 into the opening defined by the set of protrusions 70. The end portions, 64 and 64′, are thereby resiliently held in place in the openings defined by each set of protrusions, 70 and 70′.

The nose-bridge of the embodiment shown in FIGS. 5 through 8 may also be incorporated into existing swimming goggles. In such an embodiment, the nose-bridge would also include two connectors attachable to the inner ends of the eyepieces of existing swimming goggles. Each connector would define the hole through which the strap is slidably passable, and would define the set of protrusions near each hole. For example, the connectors could be attached to preexisting swimming goggles by attaching, through various means, the connectors to holes on the inner ends of the eyepieces. It will be appreciated that there are many different ways that such connectors could be attached to the inner ends of the eyepieces of existing swimming goggles.

In the embodiments shown, the strap, 22 or 62, may be made of a soft elastomer band, or any other suitable material which has an element of elasticity to it. By using a soft elastomer band, the strap conforms to the contour of a wearer's nose and acts as a cushion, resulting in a comfortable fit of the nose-bridge across the wearer's face. The elasticity of the strap allows the goggles to settle in the most comfortable position around the eye sockets of the wearer to assure a perfect waterproof fit. The friction created by such an elastic strap helps to stabilize the swimming goggles during turns and dives, which was not previously accomplished by swimming goggles incorporating string-like nose-bridges. Further, unlike swimming goggles having fixed or rigid nose-bridges that inherently create a static force towards the nose, use of an elastic strap generates a dynamic force which automatically adjusts to movements of facial muscles and therefore assures a continuous seal between the eyepieces and the face of the wearer.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. The invention includes all embodiments and variations substantially as hereinbefore described and with reference to the examples and drawings.

I claim:

1. A nose-bridge for swimming goggles having two eyepieces, each eyepiece accommodating a lens and having an inner end, each of said inner ends having a portion defining a hole therethrough, the nose-bridge comprising:
a strap for joining the inner ends of the eyepieces, said strap having first and second end portions and being slidably passable through each of the holes;
a connector for connecting said first and second end portions when said strap is passed through the holes so that said strap forms a closed loop, said connector allowing for adjustment of said strap to vary the circumference of said loop and thereby adjust the distance between the eyepieces.

2. A nose-bridge according to claim 1, wherein said connector allows for infinite adjustment of the distance between the eyepieces.

3. A nose-bridge according to claim 2, wherein said connector comprises
a portion defining at least one aperture, through which said strap is slidably passable; and
two sets of protrusions, each of said set of protrusions extending towards each other to define an opening between said set of protrusions, such that when an end portion of said strap is passed through said aperture and moved between one of said set of protrusions into one of said openings, said end portion is resiliently held in place in said opening.

4. A nose-bridge according to claim 1, wherein said strap is elastic.

5. Swimming goggles comprising:
two eyepieces, each eyepiece accommodating a lens and having an inner end, each of said inner ends having a portion defining a hole therethrough;
a strap for joining said inner ends to form a nose-bridge therebetween, said strap having first and second end portions and being slidably passable through each of said holes;
a connector for connecting said first and second end portions when said strap is passed through said holes so that said strap forms a closed loop, said connector allowing for adjustment of said strap to vary the circumference of said loop and thereby adjust the distance between said eyepieces.

6. Swimming goggles according to claim 5, wherein said connector allows for infinite adjustment of the distance between said eyepieces.

7. Swimming goggles according to claim 6, wherein said connector comprises:
a portion defining at least one aperture, through which said strap is slidably passable; and
two sets of protrusions, each of said set of protrusions extending towards each other to define an opening between said set of protrusions, such that when an end portion of said strap is passed through said aperture and moved between one of said set of protrusions into one of said openings, said end portion is resiliently held in place in said opening.

8. Swimming goggles according to claim 5, wherein said strap is elastic.

9. A nose-bridge for swimming goggles having two eyepieces, each eyepiece accommodating a lens and having an inner end, the nose-bridge comprising:
a strap for joining the inner ends of the eyepieces;
two connectors attachable to the inner ends of the eyepieces, each connector defining a hole, through which said strap is slidably passable, and a set of protrusions near each hole, each of said set of protrusions extending towards each other to define an opening between said set of protrusions, such that when an end portion of said strap is passed through one of said holes and moved between one of said set of protrusions into one of said openings, said end portion is resiliently held in place in said opening.

10. A nose-bridge according to claim 9, wherein said strap is elastic.

11. Swimming goggles comprising:
two eyepieces, each eyepiece accommodating a lens and having an inner end, each of said inner ends having a portion defining a hole therethrough;
a strap for joining said inner ends to form a nose-bridge therebetween, said strap being slidably passable through each of said holes;
a set of protrusions near each hole on said inner end, each of said set of protrusions extending towards each other to define an opening between said set of protrusion, such that when an end of said strap is passed through one of said holes and moved between one of said set of protrusions into one of said openings, said strap is resiliently held in place in said opening.

12. Swimming goggles according to claim 11, wherein said strap is elastic.
